(19) United States
(12) Patent Application Publication
(10) Pub. No.: US 2024/0352430 A1
(43) Pub. Date: Oct. 24, 2024

(54) FoF1-ATP SYNTHASE OLIGOMER

(71) Applicant: KYOTO UNIVERSITY, Kyoto (JP)

(72) Inventors: Chimari JIKO, Kyoto (JP); Yukio MORIMOTO, Kyoto (JP); Christoph GERLE, Hyogo (JP)

(21) Appl. No.: 18/687,665

(22) PCT Filed: Sep. 1, 2022
(86) PCT No.: PCT/JP2022/032925

§ 371 Date: Feb. 28, 2024

(30) Foreign Application Priority Data
Sep. 1, 2021 (JP) 2021-142046

(51) Int. Cl. C12N 9/00

(52) U.S. Cl. CPC C12N 9/00

(57) ABSTRACT

To provide: a highly purified FoF1-ATP synthase in a large quantity in an intact state; a method for producing said enzyme in a highly purified and intact state

Solution for Solving the Problem

Provided is a purified FoF1-ATP synthase oligomer. Specifically, said oligomer is a tetramer. The method for producing the FoF1-ATP synthase oligomer comprises: (1) a step for lysing, through use of a surfactant, a biological sample containing a FoF1-ATP synthase; (2) a step for performing density gradient centrifugation on the lysate obtained in step (1); and (3) a step for isolating a fraction containing the FoF1-ATP synthase. The surfactant is an anionic surfactant and/or a nonionic surfactant. The density gradient solute used in the density gradient centrifugation is sucrose.

### TECHNICAL FIELD

The present invention relates to purified FoF1-ATP (adenosine 5′-triphosphate) synthase oligomer and a method of producing the same. More specifically, it relates to FoF1-ATP synthase tetramer purified from an organ of a mammal or cultured cells of a mammal and a method of producing the same.

### TECHNICAL BACKGROUND

FoF1-ATP synthase is ubiquitously present in mitochondrial inner membrane of eukaryotic organisms, thylakoid membrane of chloroplast, and prokaryotic cell membrane, etc., and synthesizes most of the ATP (adenosine 5′-triphosphate) consumed by cells. The enzyme is present in a mammal, such as a bovine heart, as a membrane protein complex which has a molecular weight of about 600,000 and consists of 17 different subunits (non-patent documents 1 and 2). The enzyme conducts a complex energy conversion in mitochondrial inner membranes that converts electrochemical energy utilizing a proton concentration gradient formed in a respiratory chain electron transfer system into mechanical energy of rotation and further converts it to chemical energy to synthesize ATP. The Fo moiety of the enzyme is present in the membrane and serves as a path for the proton (H⁺) to pass through the membrane. Meanwhile, its F1 moiety is present outside the membrane and has catalytic activity for synthesizing or hydrolyzing ATP in the F1 moiety (non-patent document 3).

In recent studies, FoF1-ATP synthase has been shown to function as permeability transition pores (PTPs) in a mitochondrial membrane. The functional disorder of the enzyme is related to mitochondrial diseases (the general term of diseases caused by mitochondrial dysfunction) in which various clinical symptoms such as central nervous system diseases, cardiac conduction disorders, and glomerular lesions in the kidney, etc. are recognized. In particular, the dysfunction of the enzyme as PTP is believed to cause immune metabolic diseases such as non-alcoholic steatohepatitis, diabetes, and obesity etc.

Structural elucidation of FoF1-ATP synthase has been undertaken conventionally. However, since it is a very unstable enzyme, the overall structural elucidation of the enzyme is not sufficiently advanced. Thus, the structural elucidation of the entire length of the enzyme is undertaken via two-dimensional crystallization by reconstituting the purified enzyme monomer into a lipid bilayer membrane and stabilizing the enzyme (non-patent document 4). Currently, structural analysis of a porcine FoF1-ATP synthase tetramer to which IF1 (ATP synthase inhibitory factor subunit 1), an endogenous inhibitory protein of a mammalian ATP synthase, has been bound is performed using a cryo-electron microscope (non-patent document 5). As a result, the binding of IF1 to FoF1-ATP synthase was found to stabilize the enzyme tetramer. It is considered that FoF1-ATP synthase forms oligomers in biological membranes and its smallest units with functionality are tetramers.

### PRIOR ART DOCUMENTS

### Non-Patent Documents

1. Meyer B, et al., Identification of two proteins associated with mammalian ATP synthase. Molecular & Cellular Proteomics. 2007; 6:1690-1699.
2. Runswick MJ, et al., The affinity purification and characterization of ATP synthase complexes from mitochondria. Open Biology. 2013; 3:120160.
3. Ueno H, et al., ATP-driven stepwise rotation of FoF1-ATP synthase. PNAS. 2005:102:1333-1338.
4. Jiko C, et al., Bovine F1Fo ATP synthase monomers bend the lipid bilayer in 2D membrane crystals. eLife. 2015:4: e06119.
5. Gu J, et al., Cryo-EM structure of the mammalian ATP synthase tetramer bound with inhibitory protein IF1. Science. 2019:364: 1068-1075.

### SUMMARY OF THE INVENTION

### Problems to be Solved by the Invention

Since FoF1-ATP synthase is associated with mitochondrial diseases and immune metabolic diseases, etc., it is important to perform structural analysis and function elucidation of the enzymes as well as drug design targeting the enzymes. In order to do these, a highly purified enzyme with low impurity is required. However, since the enzyme is flexible and the subunit of the enzyme is extremely unstable due to its rotation, the purification of the full length of the enzyme is very difficult. In fact, the highly purified enzyme has not yet been obtained. In view of such a situation, the present invention is directed to providing a highly purified FoF1-ATP synthase in large quantities in an intact state. Furthermore, the present invention is directed to provide a method for producing the enzyme in a highly purified and intact state.

### Means for Solving the Problems

Accordingly, the objects of the present invention are achieved by the following inventions.
[1] A purified FoF1-ATP synthase oligomer.
[2] The FoF1-ATP synthase oligomer according to [1], wherein the oligomer is a tetramer.
[3] The FoF1-ATP synthase oligomer according to [1] or [2], wherein the FoF1-ATP synthase is derived from an organ of a mammal.
[4] The FoF1-ATP synthase oligomer according to [3], wherein the organ is a heart.
[5] The FoF1-ATP synthase oligomer according to [1] or [2], wherein the FoF1-ATP synthase is derived from cultured cells of a mammal.
[6] The FoF1-ATP synthase oligomer according to any of [1] to [5], wherein the FoF1-ATP synthase is derived from mitochondria.
[7] The FoF1-ATP synthase oligomer according to any of [1] to [6], wherein the mammal is a human or bovine.
[8] The FoF1-ATP synthase oligomer according to any of [1] to [6], wherein the mammal is a human.
[9] The FoF1-ATP synthase oligomer according to any of [1] to [8], which provides an independent, generally single band in clear native polyacrylamide gel electrophoresis.
[10] The FoF1-ATP synthase oligomer according to any of [1] to [9], having the activity of mitochondrial membrane permeable transition pores.
[11] The FoF1-ATP synthase oligomer according to any of [1] to [9], having ATP synthesis activity or hydrolysis activity.
[12] The FoF1-ATP synthase oligomer according to any of [1] to [11], which is reconstructed into a lipid bilayer membrane.
[12] The FoF1-ATP synthase oligomer according to [12], wherein the lipid bilayer membrane is a liposome.
[14] A preparation method of a purified FoF1-ATP synthase oligomer, comprising
   (1) a step of dissolving a biological sample comprising FoF1-ATP synthase with a surfactant:
   (2) a step of separating the solution obtained in step (1) using density gradient centrifugation; and
   (3) a step of isolating fractions containing FoF1-ATP synthase.
[15] The preparation method according to [14], wherein the density gradient solute in the density gradient centrifugation is sucrose.
[16] The preparation method according to or [15], wherein the surfactant is an anionic surfactant and/or a nonionic surfactant.
[17] The preparation method according to [16], wherein the nonionic surfactant is glycodiosgenin.
[18] The preparation method according to any of to [17], wherein the biological sample is an organ of a mammal.
[19] The preparation method according to [18], wherein the organ is a heart.
[20] The preparation method according to any of to [17], wherein the biological sample is cultured cells of a mammal.
[21] The preparation method according to any of to [20], wherein the mammal is a human or bovine.
[22] The preparation method according to any of to [20], wherein the mammal is a human.

### Effects of the Invention

According to the present invention, a highly purified FoF1-ATP synthase oligomer, particularly the enzyme tetramer, which has not yet been obtained in the prior art, is provided in large quantities in an intact state. In the method of the present invention, the structure of the protein is not destroyed in the purification process of the protein after solubilization of the biological sample by the surfactant followed by density gradient centrifugation. Thus, according to the method of the present invention, the FoF1-ATP synthase is purified without loss of its function.

### BRIEF DESCRIPTION OF DRAWINGS

FIG. 1 shows the results of gel staining with CBB of FoF1-ATP synthase tetramers and monomers derived from bovine heart obtained by centrifugation under continuous density gradient of sucrose after CN-PAGE.

FIG. 2 shows the results of gel staining with CBB of FoF1-ATP synthase tetramers and monomers derived from bovine heart obtained by centrifugation under continuous density gradient of sucrose after SDS-PAGE.

FIG. 3A shows photographs taken by a transmission electron microscope after negative staining of FoF1-ATP synthase oligomer derived from bovine heart.

FIG. 3B shows photographs taken by a transmission electron microscope after negative staining of FoF1-ATP synthase tetramer derived from bovine heart.

FIG. 3C shows photographs taken by a transmission electron microscope after negative staining of FoF1-ATP synthase monomer derived from bovine heart.

FIG. 4 shows a photograph taken by a transmission electron microscope after negative staining of reassociated FoF1-ATP synthase oligomer derived from bovine heart.

FIG. 5 shows the results of analysis by Western blotting of FoF1-ATP synthase of cultured cells derived from human (HEK293) obtained by the first centrifugation under discontinuous density gradient of sucrose after SDS-PAGE.

FIG. 6 shows the results of determination of ATP hydrolysis activity for the samples from fractions containing FoF1-ATP synthase of cultured cells derived from human (HEK293) obtained by the first centrifugation under discontinuous density gradient of sucrose.

FIG. 7 shows the results of determination of ATP hydrolysis activity in the presence or absence of oligomycin for the samples from fractions containing FoF1-ATP synthase of cultured cells derived from human (HEK293) obtained by the first centrifugation under discontinuous density gradient of sucrose.

FIG. 8 shows the results of analysis by Western blotting using antibodies to Å subunits of the samples from fractions containing FoF1-ATP synthase of cultured cells derived from human (HEK293) obtained by the second centrifugation under continuous density gradient of sucrose after SDS-PAGE.

FIG. 9 shows the results of gel staining with CBB of the samples from fractions containing FoF1-ATP synthase of cultured cells derived from human (HEK293) obtained by the second centrifugation under continuous density gradient of sucrose after CN-PAGE.

FIG. 10 shows a photograph taken by a transmission electron microscope after negative staining of FoF1-ATP synthase tetramer of cultured cells derived from human (HEK293) obtained by the second centrifugation under continuous density gradient of sucrose.

FIG. 11 shows the results of analysis by Western blotting of FoF1-ATP synthase of cultured cells derived from human (HEK293) obtained by the third centrifugation under continuous density gradient of sucrose after CN-PAGE.

FIG. 12 shows the results of determination of ATP hydrolysis activity with or without treatment to release IF1, in the presence or absence of oligomycin for the samples from the fractions containing FoF1-ATP synthase of cultured cells derived from human (HEK293) obtained by the third centrifugation under continuous density gradient of sucrose.

FIG. 13A shows photographs taken by a transmission electron microscope after negative staining of a sample from the fraction 6 of FoF1-ATP synthase of cultured cells derived from human (HEK293) obtained by the third centrifugation under continuous density gradient of sucrose.

FIG. 13B shows photographs taken by a transmission electron microscope after negative staining of a sample from the fraction 7 of FoF1-ATP synthase of cultured cells derived from human (HEK293) obtained by the third centrifugation under continuous density gradient of sucrose.

FIG. 13C shows photographs taken by a transmission electron microscope after negative staining of a sample from the fraction 8 of FoF1-ATP synthase of cultured cells derived from human (HEK293) obtained by the third centrifugation under continuous density gradient of sucrose.

FIG. 14A shows a lane configuration of gel stained with CBB of a FoF1-ATP synthase tetramer and monomer derived from bovine heart after CN-PAGE.

FIG. 14B shows scanning results of an electrophoretic gel lane by image analysis software.

FIG. 14C shows the results of analysis of the band region shown in FIG. 14B.

FIG. 15A shows a photograph taken by a transmission electron microscope after negative staining of liposomes with DMPC as constituent lipids.

FIG. 15B shows photographs taken by a transmission electron microscope after negative staining of a FoF1-ATP synthase tetramer derived from bovine heart reconstituted in DMPC-liposome.

### EMBODIMENTS FOR CARRING OUT THE INVENTION

### FoF1-ATP Synthase Oligomer

In the purified FoF1-ATP synthase oligomer of the present invention, the “purified” refers to a state thereof separated from other biological components contained in a biological sample such as proteins, lipids, and carbohydrates, etc. The purified enzyme oligomer may be in a solution or in a suspended state in a solution. The purified enzyme oligomer may comprise a stabilizer, a pH adjuster, etc. after purification, and is preferably stored at a temperature at which the activity and structure of the enzyme are maintained. As the storage temperature, the temperature is not particularly limited as long as the activity and structure of the enzyme are maintained. However, it is preferably room temperature (15 to 25° C.) or lower, and more preferably 4° C. or lower. The purified enzyme oligomer may be cryopreserved, e.g., cryopreserved at −20° C. or lower. If the enzyme oligomer is cryopreserved at −80° C., it retains its activity and structure for more than one year. Examples of the stabilizer include non-reducing disaccharides such as sucrose and trehalose, or an inert protein such as human serum albumin or bovine serum albumin.

The purity of the FoF1-ATP synthase oligomer in the total amount of protein can be determined by a biochemical verification method. Examples of biochemical verification methods include clear-native (CN)-polyacrylamide gel electrophoresis (PAGE), blue-native (BN)-PAGE, and sodium dodecyl sulfate (SDS)-PAGE. CN-PAGE and BN-PAGE migrate without denaturing proteins, and FoF1-ATP synthase oligomers can be analyzed intact. For example, a report on the analysis of mammalian mitochondria by CN-PAGE and BN-PAGE can be referred to (Meyer B, et al., Molecular & Cellular Proteomics. 2007:6:1690-1699., Krause F, et al., Biochem Biophys Res Commun. 2005:329: 583-590). The “intact” means that the FoF1-ATP synthase oligomer retains the structure present in vivo. SDS-PAGE can denature proteins and determine the molecular weight of subunits that make up FoF1-ATP synthase. The purity of the FoF1-ATP synthase oligomer can be determined from staining pattern and staining intensity by performing Western blotting and protein staining with specific immunostaining and CBB (Coomassie Brilliant Blue) after electrophoresis. Densitometers may be used for this determination.

The purity of the FoF1-ATP synthase oligomer in the total amount of protein can also be determined by a biophysical verification method. Examples of biophysical verification methods include dynamic light scattering and transmission electron microscopes. In dynamic light scattering, the particle size and distribution of particles dispersed in a solution and being in Brownian motion are calculated. If the particles are aggregates of molecules of FoF1-ATP synthase, it can be determined whether the aggregates are monodispersed or polydispersed. With a transmission electron microscope, the aggregation state of FoF1-ATP synthase can be observed directly by negative staining. The purity of the FoF1-ATP synthase oligomer can be determined, for example, based on photographs obtained by using a transmission electron microscope after adsorbing a sample containing aggregates of molecules of FoF1-ATP synthase in a copper grid, such as a carbon support membrane, and negatively staining.

The purity of purified enzyme oligomer is 80% or more, preferably 90% or more, and more preferably 95% or more in the total amount of protein during purification, based on a biochemical verification method or a biophysical verification method.

In the FoF1-ATP synthase oligomer of the present invention, an “oligomer” is a molecule in which a plurality of FoF1-ATP synthase monomers is aggregated. The oligomer provided in the present invention is a molecule of tetramer or more, specifically a tetramer, hexamer, octamer, and decamer, wherein a tetramer which is believed to be the smallest functional unit of FoF1-ATP synthase is preferably provided. The FoF1-ATP synthase may be purified in the state of an oligomer, and the enzyme disaggregated by solubilization may be returned to the oligomer by reaggregation.

In one embodiment of the invention, the FoF1-ATP synthase is derived from an organ of a mammal. Although the enzyme is purified from mammalian organs, the organ is not particularly limited as long as it contains FoF1-ATP synthase. Examples of such organs include, but are not limited to, the brain, heart, pancreas, liver, and kidney, etc., preferably the heart.

In one embodiment of the invention, the FoF1-ATP synthase is derived from cultured cells of a mammal. The cultured cells may be primary cultured cells isolated from an organ or subcultured cells. Human cardiomyocytes are exemplified as such cells. The cultured cells of a mammal may be strain-derived cells. Examples of such cells are HEK293 cells established from human fetal kidney cells and HAP1 cells of a human cell line derived from KBM-7, which is a chronic myeloid leukemia cell line.

In one embodiment of the invention, the FoF1-ATP synthase is derived from mitochondria. Mitochondria is an organelle present in all eukaryotic cells except for red blood cells. The FoF1-ATP synthase demonstrates its function in mitochondria.

In one embodiment of the invention, the mammal is preferably human or bovine, more preferably a human. The enzymes derived from human is preferably used in order to examine the relationship between human diseases and FoF1-ATP synthase oligomers.

Whether the FoF1-ATP synthase oligomer of the present invention is highly purified or not can be confirmed using various analytical methods. For example, in CN-PAGE which can be used for analysis without denaturing the FoF1-ATP synthase oligomer, the purification of the FoF1-ATP synthase tetramer of the present invention can be confirmed if the enzyme tetramer detected by immunostaining by specific antibodies in Western blotting is detected as an independent, generally single band by protein staining in the gel by CBB. As the specific antibodies, specific antibodies to the subunits of FoF1-ATP synthase can be used. The “independent” means that, for example, in protein staining by CBB, the staining band of the FoF1-ATP synthase tetramer is separated from the band on which the other proteins were stained and detected as an independent band of the present enzyme tetramer. Thus, a highly purified FoF1-ATP synthase tetramer may be obtained by cutting out the detected band. The “generally single” means that, for example, in protein staining in the gel by CBB, staining by other proteins is generally not observed as a whole gel, and most of the staining is attributed to the FoF1-ATP synthase tetramer. The “generally single” refers to that, for example, when the entire gel is scanned with a densimeter, the staining attributed to the FoF1-ATP synthase tetramer is greater than or equal to 80%, preferably greater than or equal to 90%, and more preferably greater than or equal to 95% of the total staining.

The FoF1-ATP synthase oligomer of the present invention is purified without loss of its physiological function. Thus, the enzyme oligomer, in one embodiment, has the activity of permeability transition pores in a mitochondrial membrane. The permeability transition (PT), which is the main factor that affects cell death, is a phenomenon that causes decrease in membrane potential and mitochondrial enlargement, which is resulted from Ca²⁺-dependent permeability enhancement of the mitochondrial inner membrane, which allows molecules of about 1,500 Da to permeate the inner membrane. This phenomenon is accompanied by the degradation of the mitochondrial outer membrane. PT is carried out through an opening in a channel called a permeability transition pore (PTP). While the entity that drives the physiological process of PT has not been clarified for over 60 years, it has been demonstrated by electrophysiology analysis and genetic engineering that FoF1-ATP synthase forms channels (Urbani A, et al., Purified F-ATP synthase forms a Ca²⁺-dependent high-conductance channel matching the mitochondrial permeability transition pore. Nat Commun. 2019:10: 4341). The activity of permeability transition pores in a mitochondrial membrane with such functionality can be assessed by using a cationic fluorescent dye (membrane potential probe), and for example, determining the potential difference by a flow cytometer or a microplate spectrophotometer. In addition, the activity of permeability transition pores in a mitochondrial membrane can be assessed using electrophysiological techniques such as ion leak conductance and patch clamping.

The FoF1-ATP synthase oligomer of the present invention has, in one embodiment, ATP synthesis activity or hydrolysis activity. These activities can be determined by methods well known to those skilled in the art. For example, ATP synthesis activity can be determined by using a luminescent reaction of the luciferin/luciferase system to determine the generated ATP. In addition, ATP hydrolysis activity can be determined based on absorbance changes at 340 nm by conjugating the hydrolysis reaction of ATP and the oxidation reaction from NADH (nicotinamide adenine dinucleotide) to NAD⁺ in the presence of phosphoenolpyruvic acid, pyruvate kinase, and lactate dehydrogenase.

The purified FoF1-ATP synthase oligomer of the present invention can be reconstituted into a lipid bilayer membrane. The lipid may be a phospholipid that is amphiphilic. To “reconstitute” is to incorporate a highly pure, purified FoF1-ATP synthase oligomer into a lipid bilayer membrane. It is inferred that the Fo moiety of the enzyme is incorporated into the lipid bilayer membrane and the FI moiety of the enzyme is located outside the lipid bilayer membrane. The FoF1-ATP synthase oligomer reconstituted into the lipid bilayer membrane is stable for a long period of time even at room temperature, in comparison with the FoF1-ATP synthase oligomer which is free and present in a solution. The term “stable” means that the structural change of the FoF1-ATP synthase oligomer is small and that the activity of permeability transition pores in a mitochondrial membrane and the ATP synthesis activity or hydrolysis activity of the enzyme oligomer are maintained.

The reconstitution of the FoF1-ATP synthase oligomer into the lipid bilayer membrane can be carried out by way of the following example. That is, after solubilizing the lipid component with a surfactant or an organic solvent, such as or chloroform, the surfactant or organic solvent is removed and the formed lipid membrane is stirred in a buffer to obtain a lipid bilayer membrane. By adding the enzyme oligomer thereto and incorporating it into a lipid bilayer membrane, the enzyme oligomer reconstituted in a lipid bilayer membrane can be prepared. The enzyme oligomers that are not incorporated into the lipid bilayer membrane can be removed by dialysis with a membrane filter etc., size exclusion chromatography, and centrifugation, etc.

In one embodiment of the invention, the lipid bilayer membrane is a liposome. Liposomes are vesicles having at least one lipid bilayer membrane, which comprises amphiphilic phospholipid such as phosphatidylcholine. The phospholipid may include other lipids such as phosphatidylethanolamine.

The reconstructed enzyme oligomer may contain additives such as pH adjusters and stabilizers, etc. Examples of the stabilizers include, but are not limited to, cholesterol, distearoyl phosphatidylglycerol sodium, tocopherol, and white sugar, etc.

### Preparation Methods of FoF1-ATP Synthase Oligomer

In the preparation methods of the FoF1-ATP synthase oligomer of the present invention, the biological sample comprising the enzyme is dissolved by a surfactant. In one embodiment of the invention, the biological sample is an organ of a mammal. The organ is not particularly limited as long as it contains FoF1-ATP synthase. Examples of such organs include, but are not limited to, the brain, heart, pancreas, liver, and kidney, etc., preferably the heart.

In one embodiment of the invention, the biological sample may be cultured cells of a mammal. The cultured cells may be primary cultured cells isolated from an organ or subcultured cells. Human cardiomyocytes are exemplified as such cells. The cultured cells of a mammal may be strain-derived cells. Examples of such cells are HEK293 cells established from human fetal kidney cells and HAPI cells of a human cell line derived from KBM-7, which is a chronic myeloid leukemia cell line. The cultured cells may be genetically modified cells or genome-edited cells.

The mammal is not particularly limited, although it is preferably a human or bovine, more preferably a human.

A mammalian organ or cultured cells as a biological sample, may be homogenized in a buffer containing a protease inhibitor prior to dissolution with a surfactant to increase the extraction efficiency of the FoF1-ATP synthase. Examples of the surfactant used to dissolve the biological sample can include an anionic surfactant or a nonionic surfactant. Examples of anionic surfactants include sodium cholate and sodium deoxycholate, which are suitable for solubilization of membrane proteins, and sodium deoxycholate is preferable. Examples of non-ionic surfactants include octyl β-D-glucopyranoside, glycol-diosgenin (GDN), lauryl maltose neopentyl glycol (LMNG), N-decyl-β-D-maltopyranoside (DM), N-octanoyl-N-methylglucamine, and N-nonanoyl-N-methylglucamine, etc., which are suitable for solubilization of membrane proteins, although GDN, LMNG, and DM are preferable, and GDN is more preferable. The concentration of the surfactant used during dissolution of the biological sample is preferably from 0.01 to 3% (w/v) and more preferably from 0.05 to 2% (w/v). These surfactants may be used alone or in coexistence of different surfactants.

In the preparation method of the FoF1-ATP synthase oligomer of the present invention, the lysate of the biological sample is subjected to density gradient centrifugation. The density gradient may be continuous or discontinuous, but a condition under which the FoF1-ATP synthase oligomer can be highly purified can be selected. Examples of the solutes in density gradient centrifugation include salts such as cesium chloride, cesium trifluoroacetate, sodium bromide, and potassium bromide, Percoll, and sucrose, etc., preferably sucrose. The density gradient centrifugation of a lysate of a biological sample may be carried out multiple times in order to increase the purity of the enzyme oligomer.

The pH of a buffer used to dissolve a biological sample and for density gradient centrifugation is preferably from 7.0 to 7.8, more preferably from 7.2 to 7.4, and most preferably 7.3. Temporary deviation in pH of a buffer from a preferred range can be tolerable, as long as it is possible to solubilize the FoF1-ATP synthase oligomer from the biological sample. In addition, in cases where its effect on the stability of the FoF1-ATP synthase oligomer is little, a deviation in the pH from its range can also be tolerable.

After the density gradient centrifugation, the fraction containing FoF1-ATP synthase can be confirmed by fractionating each liquid layer of the centrifuge tube with a pump etc., and analyzing the fraction containing the enzyme by electrophoresis etc.

### EXAMPLES

The present invention will now be described by way of examples. However, the present invention is not limited to the following examples.

### Example 1

### Purification of FoF1-ATP Synthase Oligomer from Bovine Heart Recovery of Mitochondrial Inner Membrane

The bovine heart immediately after slaughtered was finely cut and 600 g thereof were homogenized at 11,000 rpm for 10 minutes using polytron, while cooling ice, in the presence of 350 mL of 0.2 M sodium phosphate (pH 7.4), 2300 mL of deionized water, and PMSF (phenylmethylsulfonyl fluoride). The resulting cardiac homogenate was centrifuged at 1,400×g for 20 minutes and the supernatant was filtered with gauze to remove the oil. The resulting filtrate was centrifuged at 3,000×g for 25 minutes, and the residue was suspended in 40 mM HEPES/NaOH (pH 7.3) containing 5 mM EGTA (ethylene glycol tetraacetic acid)/DTT (dithiothreitol)/MgCl₂ and 0.5 mM ADP (adenosine diphosphate) and homogenized 12 times with a Dounce type homogenizer. The resulting homogenate was centrifuged at 100,000×g for 30 minutes to give the mitochondrial inner membrane as residue.

### Solubilization of FoF1-ATP Synthase

The concentration of the mitochondrial inner membrane was adjusted to 560 g/L and after confirming that the pH was nearly 6.8, sodium deoxycholate at a final concentration of 11% (w/v) and DM at a final concentration of 0.4% (w/v) were added. Immediately thereafter, the powder of KCl, followed by GDN at a final concentration of 0.1% (w/v) was added. This was centrifuged at 176,000×g for 40 minutes, and the supernatant was filtered with gauze. The resulting filtrate has pH of about 6.0 and contains the solubilized FoF1-ATP synthase.

### The First Centrifugation under Discontinuous Density Gradient of Sucrose

16 mL of a solution of sucrose (2.3 M) was added to the bottom of a centrifuge tube (2.9 cm in diameter, 10.3 cm in length) and 24 mL of a solution of sucrose (1.6 M) was gently placed thereon to create a discontinuous density gradient of sucrose. Sucrose was dissolved in 40 mM HEPES/NaOH (pH 7.3) containing 5 mM MgCl₂/EGTA/DTT, 0.5 mM ADP, 100 mM KCl, 0.1% (w/v) DM, and 0.04% (w/v) GDN. A solution of solubilized FoF1-ATP synthase was placed on the solution of sucrose at the top and ultracentrifuged at 4° C., 176,000×g for about 42 hours. After the ultracentrifugation, the solution was dispensed by a pump from the bottom of the centrifuge tube into a test tube. For biochemical verification of FoF1-ATP synthase, samples were taken from each test tube, and 3-12% CN-PAGE and 10-20% SDS-PAGE were carried out. As a result of electrophoresis, fractions containing the FoF1-ATP synthase were collected and the concentrations of sucrose in the fractions were determined. The protein solution was diluted with 40 mM HEPES/NaOH (pH 7.3) containing 5 mM EGTA/DTT/MgCl₂, 0.5 mM ADP, 100 mM KCl, and 0.02% (w/v) GDN.

### The Second Centrifugation under Discontinuous Density Gradient of Sucrose

10 mL of a solution of sucrose (2.3 M) was added to the bottom of the centrifuge tube (2.9 cm in diameter, 10.3 cm in length), and 10 mL of a solution of sucrose (1.6 M), followed by 10 mL of a solution of sucrose (1.0 M) and finally 10 mL of a solution of sucrose (0.6 M) was gently placed thereon to create a discontinuous density gradient of sucrose. Sucrose was dissolved in 40 mM HEPES/NaOH (pH 7.3) containing 5 mM MgCl₂/EGTA/DTT, 0.5 mM ADP, 100 mM KCl, and 0.02% (w/v) GDN. The solution of FoF1-ATP synthase obtained in the first density gradient centrifugation of sucrose was gently placed at the top, and ultracentrifuged at 4° C., 176,000×g for about 21 hours. After the ultracentrifugation, the solution was dispensed by a peristaltic pump from the bottom of the centrifuge tube into a test tube. For biochemical verification of FoF1-ATP synthase, samples were taken from each test tube, and 3-12% CN-PAGE and 10-20% SDS-PAGE were carried out. As a result of electrophoresis, fractions containing the FoF1-ATP synthase were collected.

### Dilution

In order to remove sucrose from samples of FoFI-ATP synthase obtained by the second centrifugation under discontinuous density gradient of sucrose, a solution of 40 mM HEPES/NaOH (pH 7.3) containing 5 mM MgCl₂/EGTA/DTT, 0.5 mM ADP, 100 mM KCl, and 0.02% (w/v) GDN was gently mixed.

### Separation of FoF1-ATP Synthase Tetramer by Centrifugation under Continuous Density Gradient of Sucrose

A continuous density gradient solution of 34% (w/v) to 68% (w/v) sucrose was prepared using a gradient master (a generator of continuous density gradient for ultracentrifuge tubes). Sucrose was dissolved in 40 mM HEPES/NaOH (pH 7.3) containing 5 mM MgCl₂/EGTA/DTT, 0.5 mM ADP, and 0.02% (w/v) GDN. The sample of FoF1-ATP synthase wherein sucrose had been removed by dilution was gently placed onto a density gradient solution of sucrose and ultracentrifuged at 4° C., 141,000×g for about 45 hours. After the ultracentrifugation, 2 mL each of the solutions was dispensed from the bottom of the centrifuge tubes into a test tube by a pump. Each dispensed fraction (test tube) was assigned a number in the order of collection. For biochemical verification of FoF1-ATP synthase, 5 μL of the sample was taken from each fraction (test tube), and 3-12% CN-PAGE and 10-20% SDS-PAGE were carried out. In one example, about 10 mg of FoF1-ATP synthase tetramer was purified from 1 kg of bovine heart.

### Separation of FoF1-ATP Synthase Monomer by Centrifugation Under Continuous Density Gradient of Sucrose

A continuous density gradient solution of 34% (w/v) to 68% (w/v) sucrose was prepared using a gradient master. Sucrose was dissolved in 40 mM HEPES/NaOH (pH 7.3) containing 5 mM MgCl₂/EGTA/DTT, 0.5 mM ADP, and 0.02% (w/v) LMNG. The sample of FoF1-ATP synthase wherein sucrose had been removed by dilution and LMNG was added to a final concentration of 0.5% (w/v) was gently placed onto a density gradient solution of sucrose and ultracentrifuged at 4° C., 141,000×g for about 45 hours. After the ultracentrifugation, 2 mL each of the solutions was dispensed from the bottom of the centrifuge tubes into a test tube by a pump. Each dispensed fraction (test tube) was assigned a number in the order of collection. For biochemical verification of FoF1-ATP synthase, 5 μL of the sample was taken from each fraction (test tube), and 3-12% CN-PAGE and 10-20% SDS-PAGE were carried out.

### Gel Staining

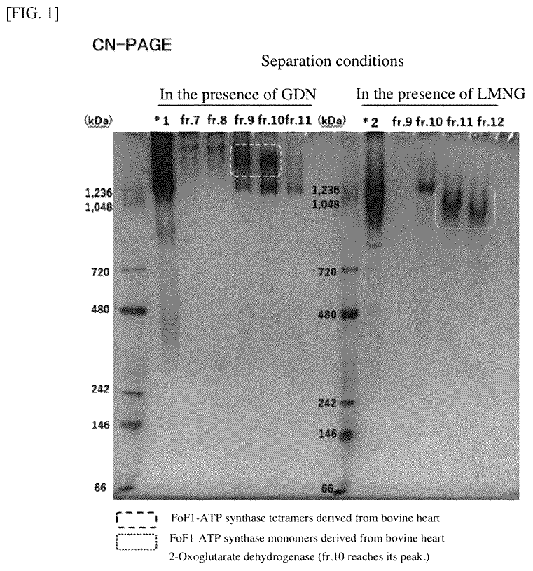

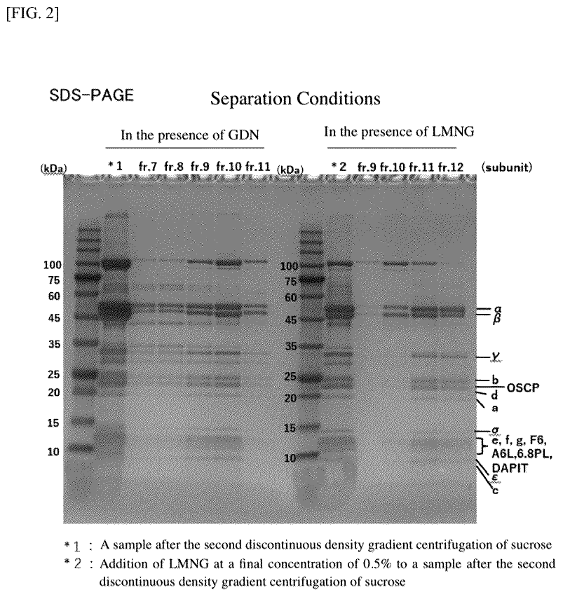

The results of analysis by CN-PAGE of FoFI-ATP synthase obtained by centrifugation under continuous density gradient of sucrose are shown in FIG. 1, and their results of analysis by SDS-PAGE are shown in FIG. 2. Gel staining was carried out with CBB (Coomassie Brilliant Blue). In either figure, a staining image of molecular weight markers (66 to 1,236 kDa) is included. As the molecular weight markers, NativeMARK® Unstained Protein Standard (Thermo Fisher Scientific) was used. The molecular weight markers include IgM hexamer (1,236 kDa), IgM pentamer (1,048 kDa), apoferritin band 1 (720 kDa), apoferritin band 2 (480 kDa), B-phycoerythrin (242 kDa), lactate dehydrogenase (146 kDa), bovine serum albumin (66 kDa), and soybean trypsin inhibitor (20 kDa).

In CN-PAGE (FIG. 1), if GDN is present as a surfactant in the density gradient solution of sucrose, FoF1-ATP synthase tetramers present at positions surrounded by dashed rectangles in the electrophoretic image of FIG. 1 were separated from the other stained bands and detected as independent, generally single bands in the samples collected from fractions 9 and 10. Thus, it was confirmed that the enzyme tetramer had been purified. The migration distance of the enzyme tetramer is shorter than that of the molecular weight marker of 1,236 kDa when compared to the migration position of the molecular weight marker in the left end lane. Thus, the molecular weight of the enzyme tetramer is shown to be greater than 1,236 kDa. On the other hand, if LMNG is present as a surfactant in the density gradient solution of sucrose, bands of the present enzyme monomer were observed in the samples collected from fractions 11 and 12. These results indicate that the presence of GDN is important for the purification of FoF1-ATP synthase tetramers by centrifugation under continuous density gradient of sucrose. Even when both GDN and LMNG were used as surfactants, 2-oxoglutaric acid dehydrogenase constituting citric acid circuit was observed in the samples collected from fractions 9 and 10 with fraction 10 as a peak top.

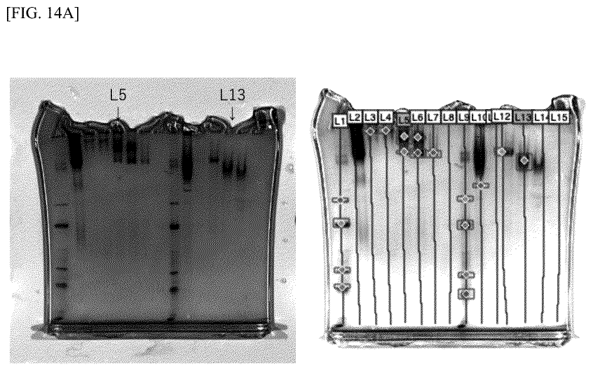

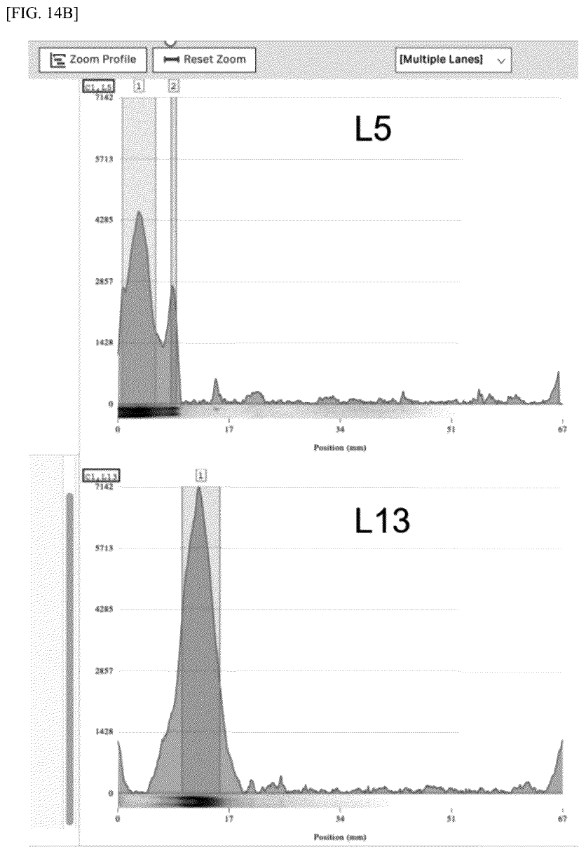

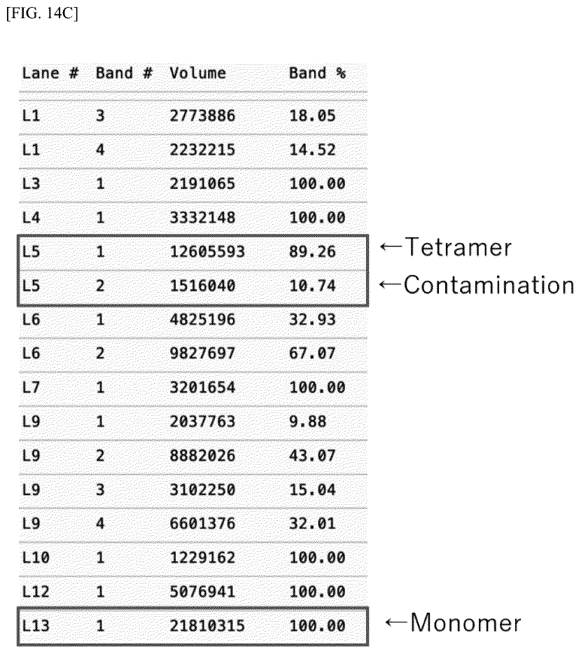

After CN-PAGE, a staining image (FIG. 1) obtained by CBB staining of gel was analyzed using the image analysis software of ImageQuantTL10.1 (Cytiva) to determine the purity of FoF1-ATP synthase. FIG. 14A shows the lanes in the gel where samples are added. Lane 5 (L5) and lane 13 (L13) in FIG. 14A were analyzed. The L5 and L13 correspond, respectively to fraction 9 (fraction of FoF1-ATP synthase tetramer) and fraction 11 (fraction of FoF1-ATP synthase monomer) in FIG. 1. FIG. 14B shows the results of scanning of L5 and L13. FIG. 14C shows the results of the analysis of the band region shown in FIG. 14B. In FIG. 14C, L5 (Band #1) corresponds to FoF1-ATP synthase tetramer, and the purity of Band #1 in L5 was 89.26%. On the other hand, the impurity (Band #2) in L5 is 10.74%. That is, the FoF1-ATP synthase tetramer has been shown to be highly purified.

In SDS-PAGE (FIG. 2), the staining images of the samples collected from fractions 9 and 10 fractionated in the presence of GDN and those of the samples collected from fractions 11 and 12 fractionated in the presence of LMNG were essentially identical. In addition, these staining images were also essentially coincident with the staining images (lanes *1 and *2) of the samples obtained as fractions containing FoF1-ATP synthase in the second centrifugation under discontinuous density gradient of sucrose. That is, in a staining image in SDS-PAGE, α, β, γ, δ, ϵ, a, b, c, d, e, f, g, A6L, F6, OSCP (oligomycin sensitivity conferral protein), DAPIT, and 6.8 PL (6.8 kDa protein), which are the subunits constituting the FoF1-ATP synthase were confirmed. These results showed that the FoF1-ATP synthase separated by the second centrifugation under discontinuous density gradient of sucrose was in a highly purified state. In addition, the FoF1-ATP synthase tetramer was shown to be aggregates of the present enzyme monomer.

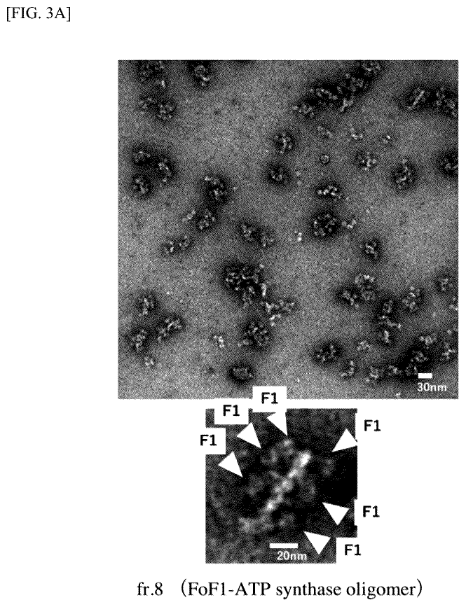

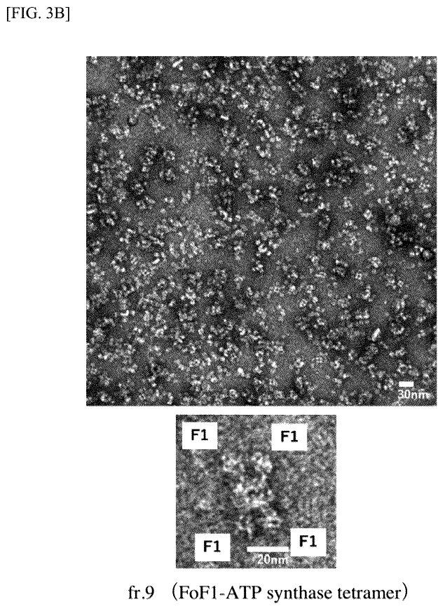

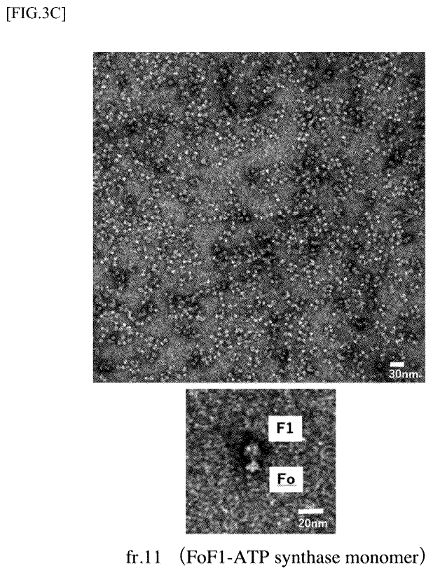

When the sample collected from fraction 8 fractionated in the presence of GDN was negatively stained and observed with a transmission electron microscope, it was shown that there were the enzyme oligomers larger than the FoF1-ATP synthase tetramer (FIG. 3A). In the enlarged photograph in the lower part of FIG. 3A, an aggregate containing six F1 moieties of the enzyme was confirmed, and it was confirmed to be a FoF1-ATP synthase hexamer. The arrow in the photograph in the lower part of FIG. 3A points to the F1 moieties. The samples collected from fraction 9 fractionated in the presence of GDN was observed in the same way. As a result, the presence of a FoF1-ATP synthase tetramer was confirmed (FIG. 3B). In the enlarged photograph in the lower part of FIG. 3B, four F1 moieties of the enzyme are confirmed, and clearly it is a tetramer. On the other hand, when a sample collected from fraction 11 fractionated in the presence of LMNG was negatively stained and observed with a transmission electron microscope, the FoF1-ATP synthase present in fraction 11 was shown to be a monomer (FIG. 3C). The Fo and F1 in the photograph in the lower part of FIG. 3C are the Fo moiety and F1 moiety, respectively, of the FoF1-ATP synthase, which were confirmed to be a monomer.

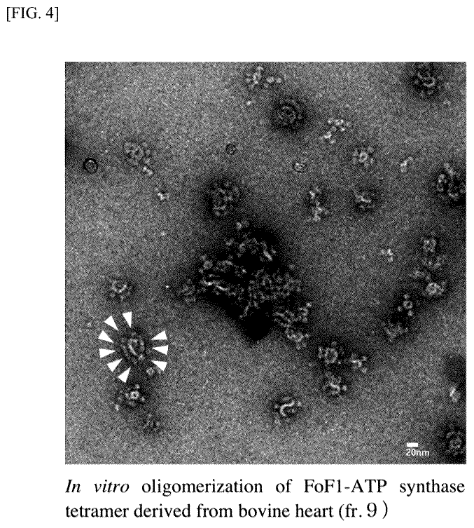

The FoF1-ATP synthase tetramer contained in fraction 9 fractionated in the presence of GDN showed that the enzyme tetramer solubilized in the presence of GDN returns to a FoF1-ATP synthase oligomer greater than the enzyme tetramer in vitro by replacing the solvent in which the enzyme was dissolved with other solvents (FIG. 4). FIG. 4 is a photograph of a FoF1-ATP synthase oligomer negatively stained and taken by a transmission electron microscope, wherein the arrows indicate the FoF1-ATP synthase oligomer. This result indicates that the purified FoF1-ATP synthase tetramer retains its in vivo state to a high degree and is therefore capable of reassociating into oligomers larger than tetramer.

### Example 2

### Purification of FoF1-ATP Synthase Oligomer from Cultured Cells Derived from Human

### Recovery of Mitochondrial Inner Membrane and Solubilization of FoF1-ATP Synthase

HEK293 cells (82 g) established from human fetal kidney cells were made 10% homogenate in 20 mM HEPES/NaOH (pH 7.3) containing 2.5 mM MgCl₂/EGTA, 250 mM sucrose and PMSF, and mitochondrial inner membrane (6.4 g) was obtained by a method similar to Example 1. The concentration of the mitochondrial inner membrane was adjusted to 490 g/L, and GDN at a final concentration of 2% (w/v) and KCl at a final concentration of 72 g/L were added, solubilized while ice-cooled for 1.5 hours, centrifuged at 184,000×g, and supernatant was obtained. The mitochondrial inner membrane was recovered three times independently, and the recovery rate of the mitochondrial inner membrane relative to the cell weight was about 10% on average.

### The First Centrifugation under Discontinuous Density Gradient of Sucrose

16 mL of a sucrose solution (2.3 M) was added to the bottom of a centrifuge tube (2.9 cm diameter, 10.3 cm length) and 24 mL of a solution of sucrose (1.6 M) was gently placed thereon to create a discontinuous density gradient of sucrose. Sucrose was dissolved in 40 mM HEPES/NaOH (pH 7.3) containing 5 mM MgCl₂/EGTA/DTT, 0.5 mM ADP, 100 mM KCl, and 0.05% (w/v) GDN. A supernatant containing solubilized FoF1-ATP synthase was placed on the solution of sucrose at the top and ultracentrifuged at 4° C., 176,000×g for about 42 hours. After the ultracentrifugation, the solution was dispensed by a peristaltic pump from the bottom of the centrifuge tube into a test tube (fraction). Each dispensed fraction (test tube) was assigned a number in the order of collection. The volumes of fractions 1 to 3 were each 4 mL, and those of fractions 4-1, 4-2, 5-1, 5-2, 6-1, 6-2, and 7-1 were each 2 mL.

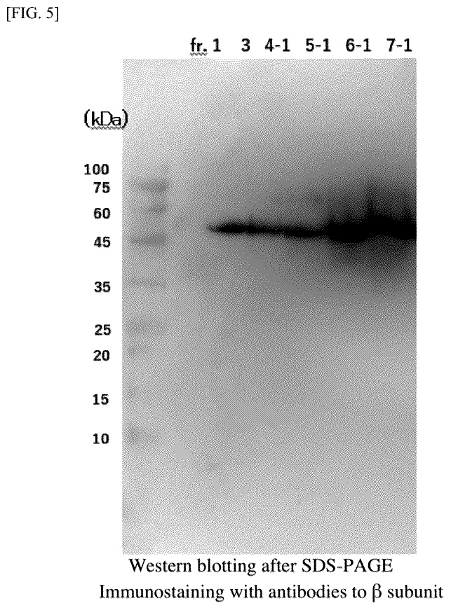

For biochemical verification of FoF1-ATP synthase, 5 uL of the sample was taken from each fraction, 10-20% SDS-PAGE was carried out, and FoF1-ATP synthase was detected by Western blotting. That is, after the SDS-PAGE, it was transferred to a poly vinylidene fluoride (PVDF) membrane and immunostaining was carried out with antibodies to the B subunit of the FoF1-ATP synthase. The results are shown in FIG. 5, which indicates the presence of FoF1-ATP synthase in fractions 3, 4-1, 5-1, 6-1, and 7-1.

### Measurement of ATP Hydrolysis Activity

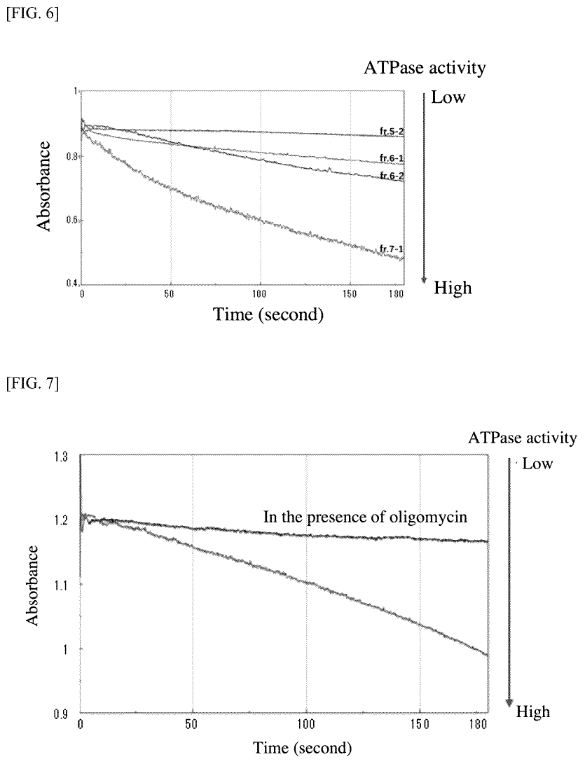

ATP hydrolysis activity of the fractions containing cultured cell FoF1-ATP synthase obtained by the first centrifugation under discontinuous density gradient of sucrose was determined. That is, the hydrolysis reaction of ATP with FoF1-ATP synthase was determined at 20° C. as change in absorbance at 340 nm by oxidation from NADH to NAD⁺ conjugated with this reaction. The results are shown in FIG. 6, which indicates that fraction 7-1 has a higher ATP hydrolysis activity compared to fractions 5-2, 6-1, and 6-2, and the FoF1-ATP synthase contained in fraction 7-1 is a FoF1-ATP synthase that has no endogenous inhibitory protein IFI bound or an ATP synthase only with F1 domain.

ATP hydrolysis activity of the samples contained in fraction 6-1 was determined in the same manner as described above in the presence or absence of oligomycin. The results are shown in FIG. 7. Since oligomycin binds to the Fo moiety of the FoF1-ATP synthase and inhibits ATP synthesis and hydrolysis, it is proved to be based on FoF1-ATP synthase if ATP synthesis and hydrolysis of the enzyme were inhibited. In FIG. 7, the ATP hydrolysis activity of the sample contained in fraction 6-1 is inhibited in the presence of oligomycin, indicating that FoF1-ATP synthase is contained in fraction 6-1.

### The Second Centrifugation under Continuous Density Gradient of Sucrose

A continuous density gradient solution of 34% (w/v) to 68% (w/v) sucrose was prepared using a gradient master (a generator of density gradient for ultracentrifuge tubes). Sucrose was dissolved in 40 mM HEPES/NaOH (pH 7.3) containing 5 mM MgCl₂/EGTA/DTT, 100 mM KCl, 0.5 mM ADP, and 0.02% (w/v) GDN. A fraction sample containing FoF1-ATP synthase in the first centrifugation under discontinuous density gradient of sucrose was gently placed on density gradient solution of sucrose and ultracentrifuged at 4° C., 141,000×g for 45 hours. After the ultracentrifugation, the solution was dispensed by a peristaltic pump from the bottom of the centrifuge tube into a test tube (fraction). Each dispensed fraction (test tube) was assigned a number in the order of collection. The volume of each fraction was 2 mL.

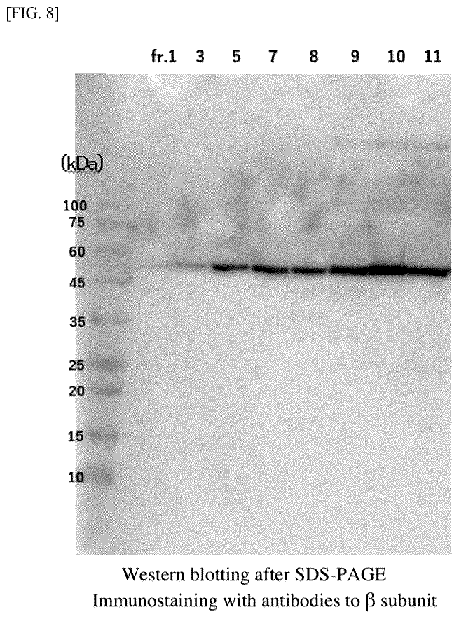

For biochemical verification of FoF1-ATP synthase, 5 μL of the sample was taken from each fraction, and 10-20% SDS-PAGE and 3-12% CN-PAGE were carried out. In SDS-PAGE, it was transfer to a PVDF membrane after gel electrophoresis, and Western blotting was carried out using antibodies to β subunits of FoF1-ATP synthase to detect FoF1-ATP synthase. The results are shown in FIG. 8, which indicates the presence of FoF1-ATP synthase in fractions 5 to 11.

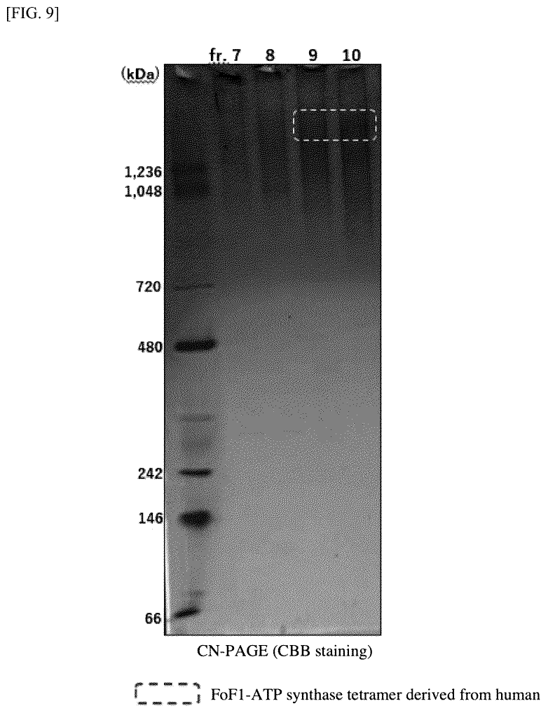

In CN-PAGE, the results of protein staining by CBB are shown in FIG. 9, from which the presence of FoF1-ATP synthase tetramer surrounded by dashed rectangle in fractions 9 and 10 was confirmed. The migration distance of the enzyme tetramer is shorter than that of the molecular weight marker of 1,236 kDa when compared to the migration position of the molecular weight marker in the left end lane. Thus, the molecular weight of the enzyme tetramer is shown to be greater than 1,236 kDa. As the molecular weight markers, the NativeMARK® Unstained Protein Standard (Thermo Fisher Scientific) described above was used.

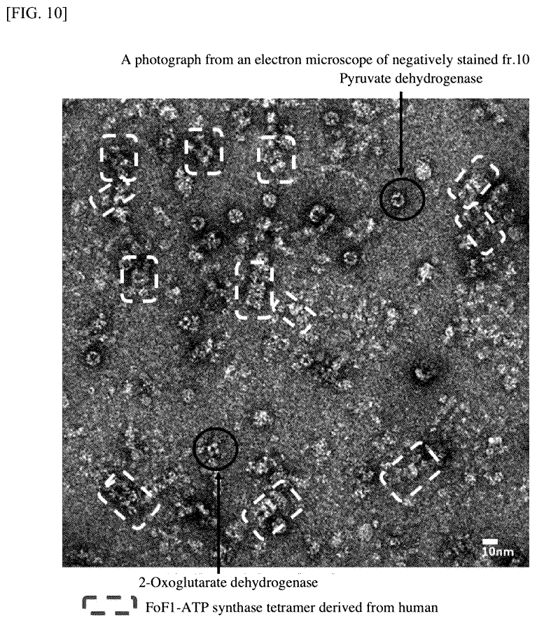

For biophysical verification of FoF1-ATP synthase, samples collected from fraction 10 were negatively stained and observed with a transmission electron microscope. The photograph is shown in FIG. 10. It was shown that FoF1-ATP synthase is present as a tetramer in the region surrounded by the dashed rectangle, although the contamination of pyruvate dehydrogenase and 2-oxoglutarate dehydrogenase in fraction 10 which is encircled in FIG. 10 was observed.

### The Third Centrifugation under Continuous Density Gradient of Sucrose

The third density gradient centrifugation of sucrose was carried out to remove the pyruvate dehydrogenase and 2-oxoglutarate dehydrogenase mixed in the fraction of FoF1-ATP synthase obtained by the second centrifugation under continuous density gradient of sucrose. A continuous density gradient solution of 20% (w/v) to 40% (w/v) sucrose was prepared using a gradient master. Sucrose was dissolved in 40 mM HEPES/NaOH (pH 7.3) containing 5 mM MgCl₂/EGTA/DTT, 50 mM KCl, 0.5 mM ADP, and 0.02% (w/v) GDN. The sample placed on the density gradient solution of sucrose is the fraction 5-2 in the first density gradient centrifugation of sucrose subjected to the second density gradient centrifugation of sucrose and also the fraction 10 fractionated in the second density gradient centrifugation. The density gradient centrifugation was carried out at 4° C., 141,000×g for 25 hours. After the ultracentrifugation, the solution was dispensed by a peristaltic pump from the bottom of the centrifuge tube into a test tube (fraction). Each dispensed fraction (test tube) was assigned a number in the order of collection. The volume of each fraction was 2 mL.

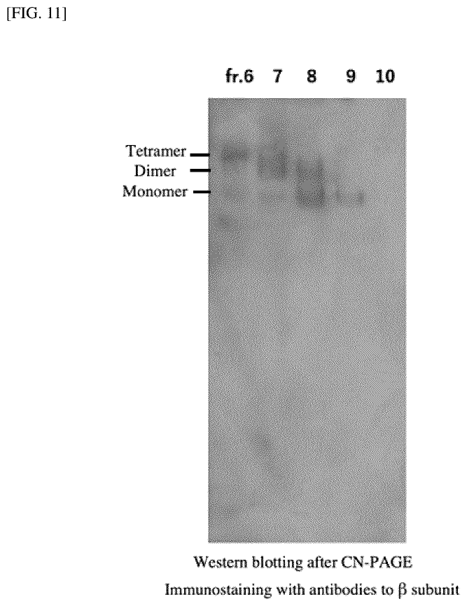

The samples from the resulting fractions 6 to 10 were subjected to CN-PAGE, transferred to PVDF membranes. Western blotting was carried out using antibodies to B subunits of FoF1-ATP synthase, and FoF1-ATP synthase was detected. The results are shown in FIG. 11, from which it was found that fraction 6 contained tetramer, fraction 7 contained tetramer and dimer, fraction 8 contained dimer and monomer, and fraction 9 contained monomer of the enzyme.

ATP hydrolysis activity of the samples collected from fraction 7 containing FoF1-ATP synthase tetramer and dimer was determined in the presence or absence of oligomycin, as in Example 1. If IF1, an endogenous inhibitory protein of mammalian ATP synthase, binds to FoF1-ATP synthase, the enzyme reaction is stopped. In order to release from FoF1-ATP synthase, samples collected from fraction 7 were mixed with 330 mM KCl and 100 mM Tris-HCl (pH 8.2). After incubation for 10 minutes at room temperature, ATP hydrolysis activity was determined. IF1 forms a dimer, binds between two FoF1-ATP synthase dimers, and stops the rotation of F1 present outside the membrane. As a result, ATP synthesis of the enzyme is stopped. Oligomycin binds to Fo of FoF1-ATP synthase and inhibits ATP synthesis.

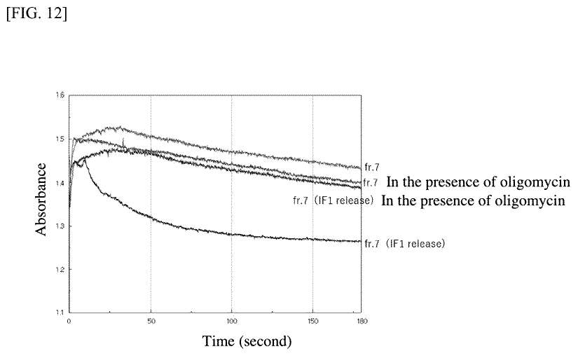

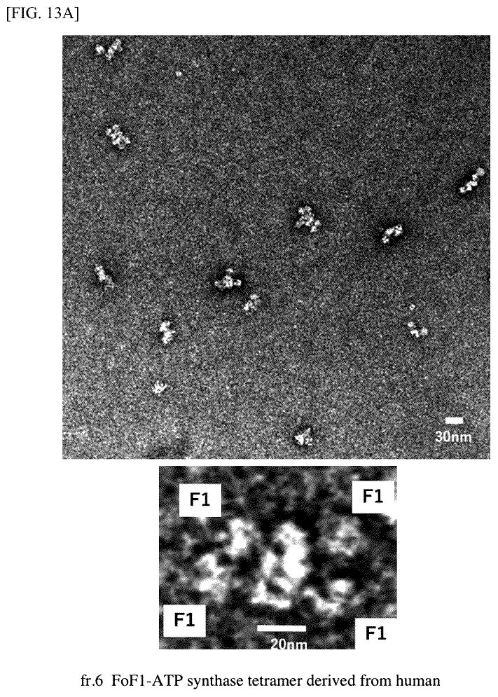

The results of the determination of ATP hydrolysis activity are shown in FIG. 12, according to which, the sample of fraction 7 did not exhibit ATP hydrolysis activity as it was. However, after the treatment to release IF1, it exhibited the activity. On the other hand, in the presence of oligomycin, it did not exhibit ATP hydrolysis activity regardless of the presence or absence of the treatment to release IF1. Thus, the ATP synthase contained in fraction 7 has Fo since it undergoes enzyme inhibition by oligomycin and has F1 since it undergoes enzyme inhibition by IF1. These results indicated the presence of FoF1-ATP synthase in said fraction 7.

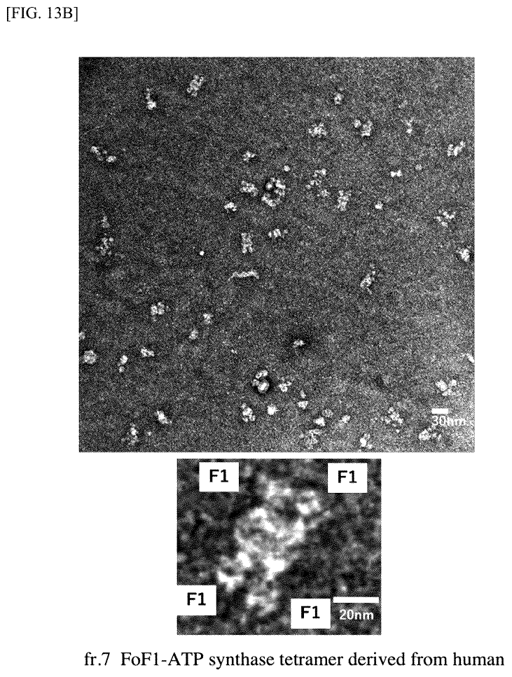

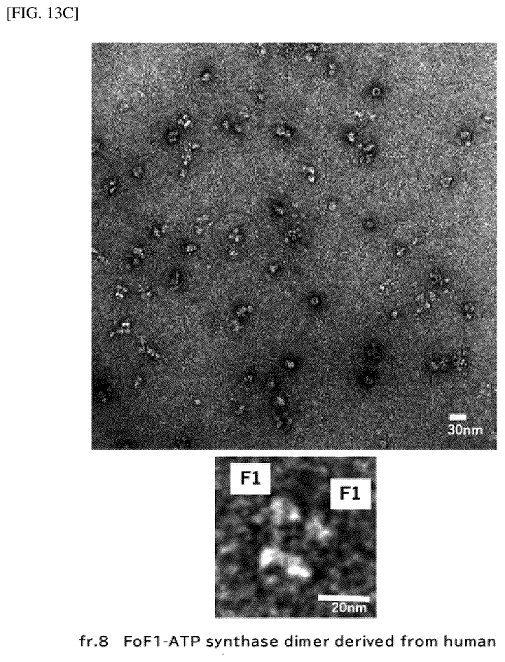

The samples collected from fractions 6, 7, and 8 obtained by the third centrifugation under continuous density gradient of sucrose in the presence of GDN as a surfactant were negatively stained and observed with a transmission electron microscope. The results are shown in FIGS. 13A, 13B, and 13C, respectively. The bottom views of FIGS. 13A, 13B, and 13C are enlarged photographs of the encircled regions in respective top views. The photographs from an electron microscope (FIG. 13A) of the sample from fraction 6, which is determined to contain FoF1-ATP synthase tetramer by Western blotting after CN-PAGE, confirmed that the enzyme tetramer was present in, for example, the encircled region in the top view. The bottom view of FIG. 13A is a further enlarged photograph, wherein four F1 moieties of the enzyme are confirmed, being clearly a tetramer.

It is determined that FoF1-ATP synthase tetramer is contained also in fraction 7. According to FIG. 13B, the present enzyme tetramer was confirmed to be present in, for example, the encircled region in the top view, similar to the sample from fraction 6. In the enlarged photograph of the bottom view of FIG. 13B, four F1 moieties of the enzyme are confirmed and the presence of the tetramer is clear. A FoF1-ATP synthase dimer is present in fraction 8, for example, a dimer is found in the encircled region in the top view of FIG. 13C. In the enlarged photograph of the bottom view of FIG. 13C, a dimer with two F1 moieties are identified. These results for fractions 6 to 8 are consistent with the results obtained by Western blotting analyses after CN-PAGE.

### Example 3

### Preparation of Proteoliposomes in which FoF1-ATP Synthase Tetramer Derived from Bovine Heart Is Reconstituted into Liposomes

### Preparation of Liposomes

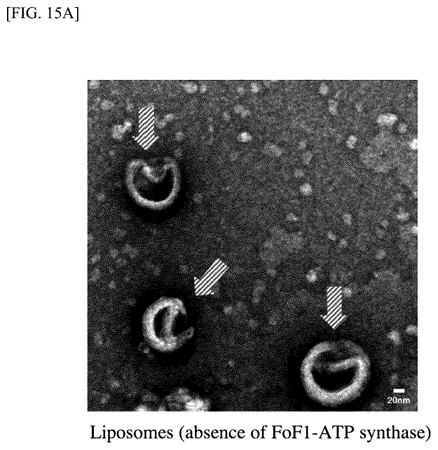

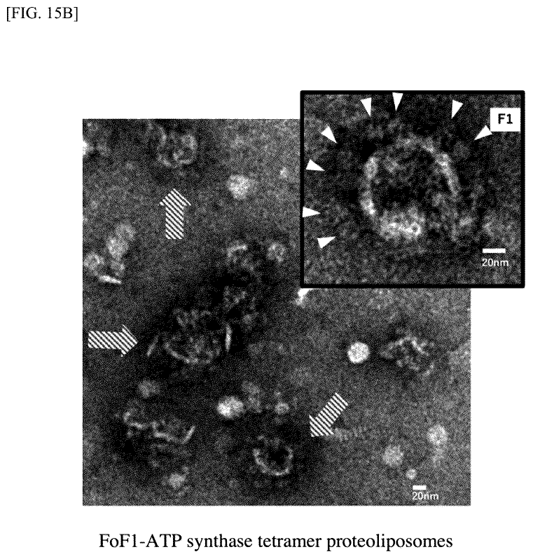

Phospholipid DMPC (1,2-dimyristoyl-sn-glycero-3-phosphocholine) was dissolved in 10% DM to make a concentration of 10 mg/mL. The DMPC solution and 10 mM HEPES (pH 7.4) containing 50 mM KCl were mixed in an equal amount. The resulting equivalent mixture was added to 20 μL of dialysis button (Hampton Research), covered with a dialysis membrane (50,000 MW cut-off), and then sealed with an O-ring. The dialysis button was placed in 10 mM HEPES (pH 7.4) containing 100 mM KCl and incubated at 37° C. until the solution turned cloudy due to the generated liposomes in the dialysis button. The resulting liposomes were negatively stained and observed with a transmission electron microscope. The results are shown in FIG. 15A, wherein the liposomes are indicated by hatched arrows. It was confirmed that lipid bilayer membranes were formed.

### Preparation of Proteoliposomes

The purified FoF1-ATP synthase tetramer derived from bovine heart obtained in Example 1 was mixed with the liposomes obtained as above to provide proteoliposomes in which the tetramer was incorporated into liposomes. The resulting proteoliposomes were negatively stained and observed with a transmission electron microscope. The results are shown in FIG. 15B, wherein the proteoliposomes are indicated by hatched arrows. The inset in FIG. 15B is an enlarged view of the proteoliposomes. In this enlarged view, the F1 moieties of the FoF1-ATP synthase are indicated by arrows, indicating that the F1 moieties of the FoF1-ATP synthase are observed to be outside the lipid bilayer membrane of the DMPC-liposome. That is, it can be seen that the natural state of the FoF1-ATP synthase, in which the Fo moiety is present inside the membrane and the F1 moiety is present outside the membrane, has been reconstituted.

1. A purified FoF1-ATP synthase oligomer.

2. The FoF1-ATP synthase oligomer according to claim 1, wherein the oligomer is a tetramer.

3. The FoF1-ATP synthase oligomer according to claim 1, wherein the FoF1-ATP synthase is derived from an organ of a mammal.

4. The FoF1-ATP synthase oligomer according to claim 3, wherein the organ is a heart.

5. The FoF1-ATP synthase oligomer according to claim 1, wherein the FoF1-ATP synthase is derived from cultured cells of a mammal.

6. The FoF1-ATP synthase oligomer according to claim 1, wherein the FoF1-ATP synthase is derived from mitochondria.

7. The FoF1-ATP synthase oligomer according to tclaim 1, wherein the mammal is a human or bovine.

8. The FoF1-ATP synthase oligomer according to claim 1, wherein the mammal is a human.

9. The FoF1-ATP synthase oligomer according to claim 1, which provides an independent, generally single band in clear native polyacrylamide gel electrophoresis.

10. The FoF1-ATP synthase oligomer according to claim 1, having the activity of mitochondrial membrane permeable transition pores.

11. The FoF1-ATP synthase oligomer according to claim 1, having ATP synthesis activity or hydrolysis activity.

12. The FoF1-ATP synthase oligomer according to claim 1, which is reconstructed into a lipid bilayer membrane.

13. The FoF1-ATP synthase oligomer according to claim 12, wherein the lipid bilayer membrane is a liposome.

14. A production method of purified FoF1-ATP synthase oligomer, comprising
(1) a step of dissolving a biological sample comprising FoF1-ATP synthase with a surfactant;
(2) a step of separating the solution obtained in step (1) using density gradient centrifugation; and
(3) a step of isolating fractions containing FoF1-ATP synthase.

15. The production method according to claim 14, wherein the density gradient solute in the density gradient centrifugation is sucrose.

16. The production method according to claim 14, wherein the surfactant is an anionic surfactant and/or a nonionic surfactant.

17. The production method according to claim 16, wherein the nonionic surfactant is glycodiosgenin.

18. The production method according to claim 14, wherein the biological sample is an organ of a mammal.

19. The production method according to claim 18, wherein the organ is a heart.

20. The production method according to claim 14, wherein the biological sample is cultured cells of a mammal.

21. The production method according to claim 18, wherein the mammal is a human or bovine.

22. The production method according to claim 18, wherein the mammal is a human.
